(19) United States
(12) Patent Application Publication
(10) Pub. No.: US 2006/0161248 A1
(43) Pub. Date: Jul. 20, 2006

(54) Medical device with leak path

(76) Inventors: Brian C. Case, Bloomington, IN (US); Charles W. Agnew, West Lafayette, IN (US)

Correspondence Address: DUNLAP, CODDING & ROGERS, P.C., P.O. BOX 16370, OKLAHOMA CITY, OK, 73113, US

(21) Appl. No.: 11/291,636

(22) Filed: Dec. 1, 2005

Related U.S. Application Data
(60) Provisional application No. 60/522,998, filed on Dec. 1, 2004.

(51) Int. Cl. A61F 2/24

(52) U.S. Cl. 623/2.1

(57) ABSTRACT

Medical devices that provide valves for regulating fluid flow through a body vessel are provided. The valves include a support frame having one or more adaptations for forming a leak path between the support frame and an interior wall of a body vessel in which the valve is implanted. A controlled amount of retrograde flow is able to flow through the leak path when the valve is implanted in a body vessel.

### CROSS-REFERENCE TO RELATED APPLICATION

This application claims priority to U.S. Provisional Application Ser. No. 60/522,998, filed on Dec. 1, 2004, the entire disclosure of which is hereby incorporated into this disclosure in its entirety.

### FIELD

The application for patent relates to medical devices. Exemplary embodiments described herein relate to valves that can be implanted in a body vessel or other suitable locations within the body of an animal, such as a human.

### BACKGROUND

Many vessels in animal bodies transport fluids from one bodily location to another. Frequently, fluid flows in a unidirectional manner along the length of the vessel. Varying fluid pressures over time, however, can introduce a reverse flow direction in the vessel. In some vessels, such as mammalian veins, natural valves are positioned along the length of the vessel and act as one-way check valves that open to permit the flow of fluid in the desired direction and close to prevent fluid flow in a reverse direction, i.e., retrograde flow. The valves can change from an open position in response to a variety of circumstances, including changes in the cross-sectional shape of the vessel and the fluid pressure within the vessel.

While natural valves may function for an extended time, some may lose effectiveness, which can lead to physical manifestations and pathology. For example, venous valves are susceptible to becoming insufficient due to one or more of a variety of factors. Over time, the vessel wall may stretch, affecting the ability of the valve members to close. Furthermore, the valve members may become damaged, such as by formation of thrombus and scar tissue, which may also affect the ability of the valve members to close. Once valves are damaged, venous valve insufficiency may be present, which can lead to discomfort and possibly ulcers in the legs and ankles.

Current treatments for venous valve insufficiency include the use of compression stockings that are placed around the leg of a patient in an effort to force the vessel walls, radially inward to restore valve function. Surgical techniques are also employed in which valves can be bypassed, eliminated, or replaced with autologous sections of veins having competent valves.

Minimally invasive techniques and instruments for placement of intraluminal medical devices have developed over recent years. A wide variety of treatment devices that utilize minimally invasive technology has been developed and includes stents, stent grafts, occlusion devices, infusion catheters and the like. Minimally invasive intravascular devices have especially become popularwith the introduction of coronary stents to the U.S. market in the early 1990s. Coronary and peripheral stents have been proven to provide a superior means of maintaining vessel patency, and have become widely accepted in the medical community. Furthermore, the use of stents has been extended to treat aneurysms and to provide occlusion devices, among other uses. Recently, valves that are implantable by minimally invasive techniques have been developed. Frequently, a valve member is attached to a support frame and provides a valve function to the device. For example, the valve member can be in the form of a leaflet that is attached to a support frame and movable between first and second positions. In a first position, the valve is open and allows fluid flow to proceed through a vessel in a first direction, and in a second position the valve is closed to prevent fluid flow in a second, opposite direction. Examples of this type of valve are described in commonly owned U.S. Pat, No. 6,508,833 to Pavcnik for a MULTIPLE-SIDED INTRALUMINAL MEDICAL DEVICE, United States Patent Application Publication No. 2001/0039450 to Pavcnik for an IMPLANTABLE VASCULAR DEVICE, and U.S. patent application Ser. No. 10/642,372, filed on Aug. 15, 2003, each of which is hereby incorporated by reference in its entirety. In other examples of valve medical devices, a tube that terminates in valve members is attached to one or more support frames to form a valve. The valve members open to permit fluid flow in a first direction in response to fluid pressure on one side of the valve members, and close to prevent fluid flow in a second, opposite direction in response to fluid pressure on opposite sides of the valve members. An example of this configuration is provided in U.S. Pat. No. 6,494,909 to Greenhalgh for AN ENDOVASCULAR VALVE, which is hereby incorporated by reference in its entirety.

Natural valves can be somewhat ‘leaky,’ allowing a relatively small quantity of fluid to flow in a reverse direction, i.e., retrograde flow, when the valve is in a closed position. It is believed that this leakiness is beneficial for several reasons. For example, it is believed that a small amount of retrograde flow limits the pooling of blood around the natural valve during periods of low pressure, which can reduce the formation of thrombus adjacent the valve members and, therefore, increase the effective lifetime of the valve.

Prior art valve devices, however, do not permit a controlled amount of retrograde flow. Indeed, most prior art valves have been designed to prevent leakage as much as possible. Accordingly, there is a need for valve devices that permit a controlled amount of retrograde flow.

### SUMMARY OF EXEMPLARY EMBODIMENTS

Medical devices comprising a valve for regulating fluid flow through a body vessel are described. The valves can be used in a variety of locations, including venous and cardiac applications, and include a leak path through which a controlled amount of retrograde flow can pass.

An implantable medical device according to one exemplary embodiment comprises a support frame having radially compressed and radially expanded configurations and a means for forming a leak path between the support frame and an interior wall of said body vessel. -A valve member is attached to the support frame and is moveable between a first position that permits said fluid flow in a first direction and a second position that substantially prevents said fluid flow in a second direction. Any suitable means for forming a leak path can be used, including one or more channels, one or more projections, one or more contours, such as a series of scallops, and one or more support wings.

An implantable medical device according to another exemplary embodiment comprises a support frame having radially compressed and radially expanded configurations. A portion of the support frame defines a channel that forms a leak path with a portion of a body vessel and allows passage of a controlled amount of fluid flow. A valve member is attached to the support frame and is moveable between first and second positions to selectively allow fluid flow through a valve orifice.

Additional understanding of the invention can be obtained with review of the description of exemplary embodiments of the invention, appearing below, and the appended drawings that illustrate exemplary embodiments.

### BRIEF DESCRIPTION OF THE DRAWINGS

FIG. 1 is a perspective view of a medical device according to a first exemplary embodiment.

FIG. 2 is a perspective view of a body vessel containing the medical device illustrated in FIG. 1.

FIG. 3 is an enlarged sectional view of the area highlighted in FIG. 2.

FIG. 4 is a perspective view of a medical device according to a second exemplary embodiment.

FIG. 5 is a perspective view of a body vessel containing the medical device illustrated in FIG. 4.

FIG. 6 is an enlarged sectional view of the area highlighted in FIG. 5.

FIG. 7 is a perspective view of a medical device according to an alternate embodiment.

FIG. 8 is a perspective view of a medical device according to an alternate embodiment.

FIG. 9 is a perspective view of a medical device according to a third exemplary embodiment.

FIG. 10 is a sectional view of a body vessel containing the medical device illustrated in FIG. 9.

FIG. 11 is a perspective view of a medical device according to a fourth exemplary embodiment.

FIG. 12 is a sectional view of a body vessel containing the medical device illustrated in FIG. 11.

FIG. 13 is a perspective view of a medical device according to a fifth exemplary embodiment.

FIG. 14 is a sectional view of a body vessel containing the medical device illustrated in FIG. 13.

### DETAILED DESCRIPTION OF EXEMPLARY EMBODIMENTS

The following provides a detailed description of exemplary embodiments. The description is not intended to limit the scope of the invention, or its protection, in any manner, but rather serves to enable those skilled in the art to practice the invention.

Medical devices that can be used in a variety of applications are provided. For example, medical devices according to exemplary embodiments comprise valves that can be used to regulate fluid flow through a body vessel. The valves can be implanted in a body vessel, or in any other suitable environment, to regulate the flow of fluid. Valves according to the invention can also be implanted in ducts, canals, and other passageways in the body, as well as cavities and other suitable locations. Valves according to exemplary embodiments of the invention can be implanted in the vessels of the vasculature, such as veins, to regulate the flow of blood through the vessels. Valves according to the invention can also be implanted in the vessels of the heart, including the aorta, as a heart valve.

As used herein, the term “implanted,” and grammatically related terms, refers to the positioning of an item in a particular environment, either temporarily, semi-permanently, or permanently. The term does not require a permanent fixation of an item in a particular position.

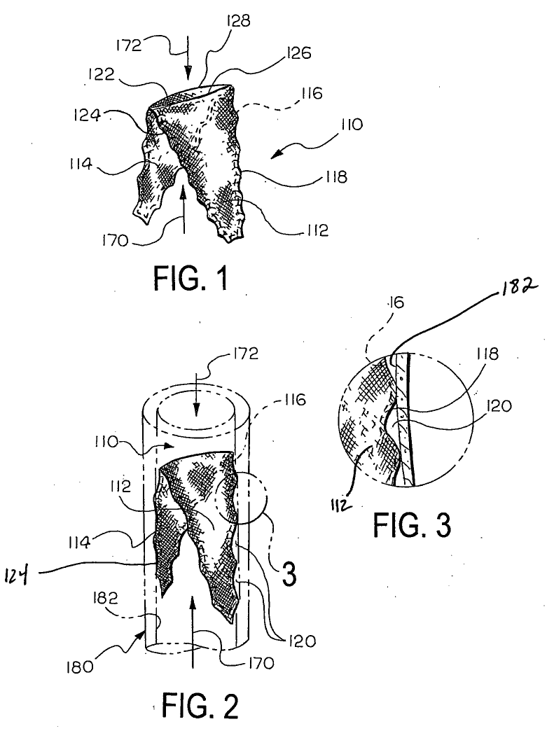

FIGS. 1 through 3 illustrate a first exemplary embodiment. The medical device according to this embodiment is a valve 110 for regulating fluid flow through a vessel. In this embodiment, the valve 110 includes two valve members 112, 114 that are attached to a support frame 116 that defines a series of scallops 118. As best illustrated in FIG. 3, a leak path 120 is formed between each scallop 118 of the support frame 116 and an interior wall 182 of the body vessel 180 in which the valve 110 is implanted. The leak path 120 provides a path through which fluid can flow without encountering the valve orifice 122 defined by the valve members 112, 114.

The valve members 112, 114 comprise a section of material. The valve members 112,114 can be formed of any suitable material, and need only be biocompatible or be able to be made biocompatible and be able to perform as described herein. The valve members 112,114 advantageously can be formed of a flexible material. Examples of suitable materials for the valve members 112, 114 include natural materials, synthetic materials, and combinations of natural and synthetic materials. Examples of suitable natural materials include extracellular matrix (ECM) materials, such as small intestine submucosa (SIS), and other bioremodellable materials, such as bovine pericardium. Other examples of ECM materials that can be used in the medical devices of the invention include stomach submucosa, liver basement membrane, urinary bladder submucosa, tissue mucosa, and dura mater. Examples of suitable synthetic materials include polymeric materials, such as expanded polytetrafluoroethylene and polyurethane. ECM materials are particularly well-suited materials for use in the valve members 112, 114 at least because of their abilities to remodel and to provide a scaffold onto which cellular in-growth can occur, eventually allowing the material to remodel into a structure of host cells.

The valve members 112, 114 can be attached to the support frame 116 in any suitable manner. As illustrated in FIG. 1, sutures 124 or other attachment members can be used to attach the valve members 112, 114 to the support frame 116. Alternatively, the valve members 112, 114 can be attached to the support frame 116 by other means for attaching, such as adhesives, heat sealing, tissue welding, weaving, cross-linking, or other suitable means for attaching. The specific means for attaching chosen will depend at least upon the materials used in the valve members 112, 114 and the support frame 116.

Free edges 126,128 of the valve members 112, 114 cooperatively define a valve orifice 122. The valve members 112, 114 are moveable between first and second positions. In the first position, the orifice 122 is open and allows fluid flow through the valve 110 in a first direction, represented by arrow 170. In the second position, the free edges 126, 128 of the valve members 112, 114 come together to close the orifice 122 and substantially prevent fluid flow through the valve 110 in a second, opposite direction, represented by arrow 172.

The leak path 120 permits a controlled amount of fluid flow through the valve 110. This controlled fluid flow can pass through the leak path 120 when the valve orifice 122 is in the open and/or closed position. It is expected, however, that fluid will flow through the leak path 120 more readily when the orifice 122 is closed because, in this configuration, the leak path 120 is the only path through which fluid can flow through the valve 110. As a result, the leak path 120 is expected to provide a path for retrograde flow to flow across the valve 110.

The support frame 116 can comprise any suitable support frame. A wide variety of support frames are known in the medical technology arts, and any suitable support frame can be utilized. The specific support frame chosen will depend on several considerations, including the nature of the valve member, the nature of the point of treatment at which the medical device will be implanted, and the medical condition for which the medical device is being used. The support frame 116 need only provide a surface to which the valve member can be attached and provide the structure needed to form the leak path 120.

The support frame 116 advantageously has radially compressed and radially expanded configurations. Such a support frame 116 can be implanted at a point of treatment within a body vessel by minimally invasive techniques, such as via delivery and deployment with an intravascular catheter. The support frame 116 can optionally provide additional function to the medical device 110. For example, the support frame 116 can provide a stenting function, i.e., exert a radially outward force on the interior wall 182 of the vessel 180 in which the medical device 110 is implanted. By including a support frame 116 that exerts such a force, a medical device according to the invention can provide both a stenting and a valving function at a point of treatment within a body vessel.

The support frame 116 can be self-expandable or balloon expandable. The structural characteristics of both of these types of support frames are known in the art, and are not detailed herein. Each type of support frame has advantages and, for any given application, one type may be more desirable the other based on a variety of considerations. For example, in the peripheral vasculature, vessels are generally more compliant and typically experience dramatic changes in their cross-sectional shape during routine activity. Medical devices for implantation in the peripheral vasculature should retain a degree of flexibility to accommodate these changes of the vasculature. Accordingly, medical devices according to the invention intended for implantation in the peripheral vasculature, such as venous valves, advantageously include a self-expandable support frame. These support frames, as known in the art, are generally more flexible than balloon-expandable support frames following deployment.

The support frame 116 can be formed of any suitable material and need only be biocompatible or able to be made biocompatible. The support frame 116 is advantageously made from a resilient material, preferably metal wire formed from stainless steel or a superelastic alloy, such as nitinol. While round wire is depicted in FIG. 1, other types, such as flat, square, triangular, D-shaped, and delta-shaped wire, may be used to form the support frame 116. Other examples of suitable materials include, without limitation, stainless steel, nickel titanium (NiTi) alloys, e.g., nitinol, other shape memory and/or superelastic materials, polymers, and composite materials. Also, resorbable and bioremodellable materials can be used, including the resorbable and bioremodellable materials described herein.

As used herein, the term “resorbable” refers to the ability of a material to be absorbed into a tissue and/or body fluid upon contact with the tissue and/or body fluid. The contact can be prolonged, and can be intermittent in nature. A number of resorbable materials are known in the art, and any suitable resorbable material can be used. Examples of suitable types of resorbable materials include resorbable homopolymers, copolymers, or blends of resorbable polymers. Specific examples of suitable resorbable materials include poly-alpha hydroxy acids such as polylactic acid, polylactide, polyglycolic acid (PGA), and polyglycolide; trimethlyene carbonate; polycaprolactone; poly-beta hydroxy acids such as polyhydroxybutyrate and polyhydroxyvalerate; and other polymers such as polyphosphazines, polyorganophosphazines, polyanhydrides, polyesteramides, polyorthoesters, polyethylene oxide, polyester-ethers (e.g., polydioxanone) and polyamino acids (e.g., poly-L-glutamic acid or poly-L-lysine). There are also a number of naturally derived resorbable polymers that may be suitable, including modified polysaccharides, such as cellulose, chitin, and dextran, and modified proteins, such as fibrin and casein.

As described above, the support frame 116 defines a series of scallops 118 for formation of the leak paths 120. Any suitable size, configuration, and number of scallops 118 can be used, and the specific size, configuration, and number used in a medical device according to a particular embodiment of the invention will depend on several considerations, including the desired quantity of fluid flow through the leak paths 120. In the illustrated embodiment, the scallops 118 are defined by a portion of the support frame 116 that has a substantially sinusoidal configuration.

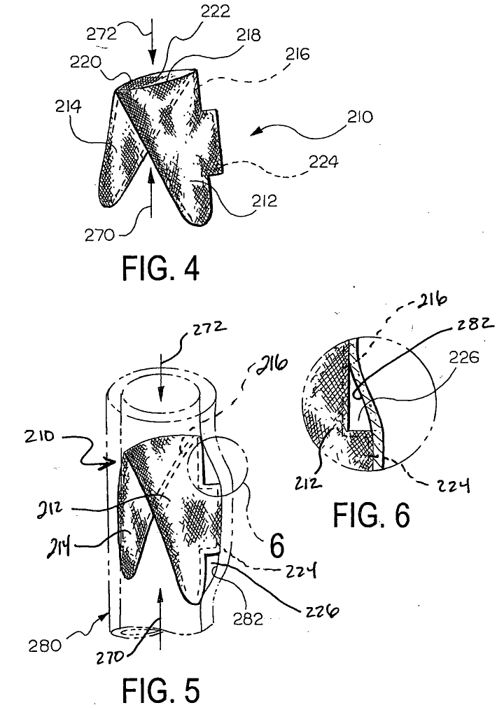

FIGS. 4 through 6 illustrate a medical device 210 according to a second embodiment of the invention. The device 210 of this embodiment is similar to the device illustrated in FIGS. 1 through 3, except as described below. Accordingly, the device 210 comprises a valve and includes two valve members 212,214 that are attached to a support frame 216. Free edges 218, 220 of the valve members 212, 214 cooperatively define a valve orifice 222. The valve members 212, 214 are moveable between first and second positions. In the first position, the orifice 222 is open and allows fluid flow through the valve 210 in a first direction, represented by arrow 270. In the second position, the free edges 218, 220 of the valve members 212, 214 come together to close the orifice 222 and substantially prevent fluid flow through the valve 210 in a second, opposite direction, represented by arrow 272.

In this embodiment, the support frame 216 defines a projection 224. As best illustrated in FIGS. 5 and 6, the projection 224 spaces an interior wall 282 of a body vessel 280 from the support frame 216 when the valve 210 is positioned within a lumen of the body vessel 280. As a result, a leak path 226 is formed. The leak path 226 permits a controlled amount of fluid flow through the valve 210, including retrograde flow 272.

The projection 224 can have any suitable shape and configuration, and can be positioned at any suitable location on the support frame 216. As best illustrated in FIGS. 4 and 5, the projection 224 can be generally rectangular in shape and be positioned across a midpoint of a length of a linear portion of the support frame 216, such as a strut. The rectangular shape of the projection 224 allows for an extended interface area between the valve 210 and the interior wall 282 of the body vessel 280, which may facilitate anchoring of the valve 210 in the body vessel 280.

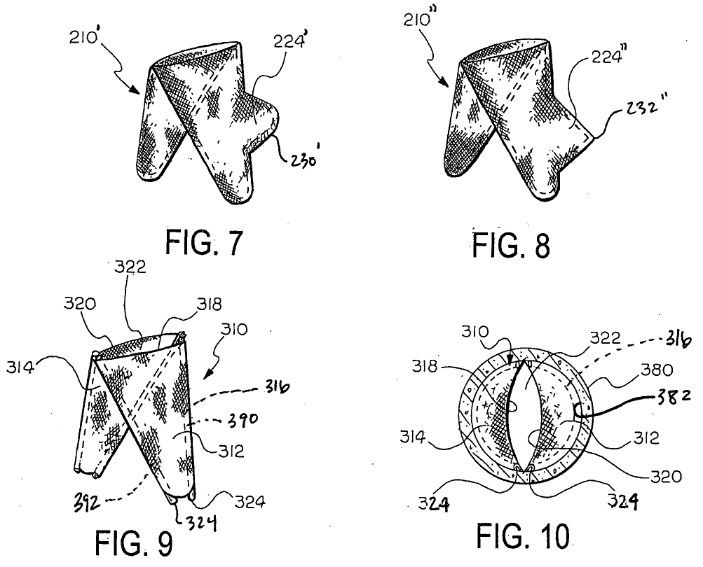

FIGS. 7 and 8 illustrate alternative projections. In the embodiment illustrated in FIG. 7, the valve 210! includes a projection 224′ that has a curvilinear surface 230′. This embodiment may be advantageous because the curvilinear surface 230′ substantially eliminates edges of the projection 224′ that interact with the vessel wall 282.

In the embodiment illustrated in FIG. 8, the valve 210″ includes a projection 224″ that has a substantially triangular shape. This embodiment may be advantageous because the substantially triangular shape may enhance anchoring of the valve 210″ in a body vessel by providing a point 232″ that can function as a barb that interacts with a wall of the body vessel. The specific shape, configuration, and position of the projection in a medical device according to a particular embodiment of the invention will depend on several considerations, including the type of body vessel in which the medical device will be implanted.

FIGS. 9 and 10 illustrate a medical device 310 according to a third exemplary embodiment of the invention. The device 310 of this embodiment is similar to the device illustrated in FIGS. 1 through 3, except as described below. Accordingly, the device 310 comprises a valve and includes two valve members 312, 314 that are attached to a support frame 316. Free edges 318, 320 of the valve members 312, 314 cooperatively define a valve orifice 322. The valve members 312, 314 are moveable between first and second positions. In the first position, the orifice 322 is open and allows fluid flow through the valve. 310 in a first direction. In the second position, the free edges 318, 320 of the valve members 312, 314 come together to close the orifice 322 and substantially prevent fluid flow through the valve 310 in a second, opposite direction.

In this embodiment of the invention, a portion of the support frame 316 defines a channel 324 that permits a controlled amount of fluid flow through the valve 310, including retrograde flow. The channel 324 cooperates with an interior wall 382 of a body vessel 380 to form a leak path.

Any suitable configuration can be used for the channel 324. Further, more than one channel can be included. The specific configuration and number chosen for any particular medical device according to the invention will depend on several considerations, including the type of support frame used and the quantity of flow needed to pass through a leak path.

In the embodiment illustrated in FIGS. 9 and 10, two struts 390, 392 of the support frame 316 include a channel 324. In this configuration, leak paths are provided on one side of the valve orifice 322 and not on the opposite side. This may be advantageous as it is expected to create an unequal distribution of retrograde flow at the valve orifice 322, which may facilitate a prevention of pooling of fluid in or around the valve 310. It is understood, however, that more or fewer channels in more or fewer struts, or other portions of a support frame, can be used without departing from the scope of the invention.

FIG. 10 illustrates the valve 310 disposed within a body vessel 380. In the illustrated embodiment, the channel 324 of the support frame 316 has a substantially ovoid cross-sectional shape. An ovoid shape is considered advantageous because it provides a relatively large void region through which fluid can flow. Any suitable cross-sectional shape can be used in the channel 324, however, and the specific cross-sectional shape used in a medical device according to a particular embodiment of the invention will depend on several considerations, including the desired quantity of fluid flow through the leak paths formed by the channel.

To facilitate fluid flow through the channel 324, it may be advantageous to include a coating on the portions of the support frame 316 that define the channel 324. Any suitable coating can be used and should be chosen to facilitate, rather than hinder, fluid flow. Examples of suitable coatings include non-thrombogenic and thromboresistant coatings, such as heparin and suitable heparin-containing compounds and mixtures. Of course, any coating having desirable properties can be used.

In this embodiment, the valve members 312, 314 are attached to the support frame 316 in a manner that does not significantly obstruct fluid flow through the channel 324. As illustrated in FIGS. 9 and 10, the valve members 312, 314 can be attached to the support frame 316 without sutures. Suture alternatives such as adhesives, heat sealing, tissue welding, weaving, cross-linking, or any other suitable means for attaching the valve members 312, 314 to the support frame 316 can be used. The specific means for attaching chosen will also depend upon the materials used in the valve members 312, 314 and the support frame 316.

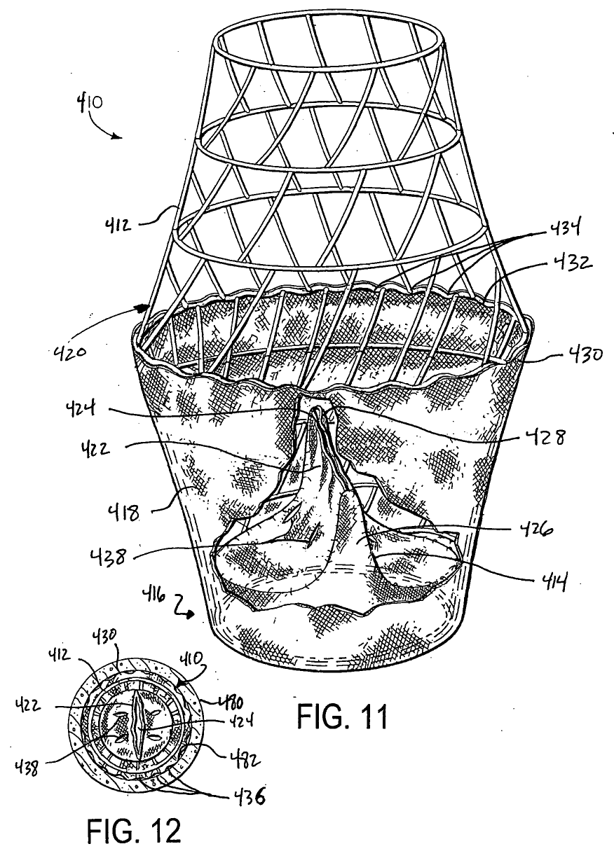

FIGS. 11 and 12 illustrate a medical device 410 according to a fourth exemplary embodiment. In this embodiment, the medical device 410 is a valve for regulating fluid flow through a body vessel. The valve includes first 412 and second 414 support frames. The first support frame 412 is a wire frame member and the second support frame 414 is a solid circumferential member, although any suitable support frame can be used for each of the support frames 412, 414. The second support frame 414 is disposed within the first support frame 412 at an end portion 416. A tubular graft member 418 is disposed on an external side 420 of the first support frame 412 and inverted into a space between the first 412 and second 414 support frames.

A first end 422 of the graft member 418 terminates in a valve orifice 424 that is supported by first 426 and second 428 upstanding arms formed by the second support frame 414. The valve orifice 424 opens and closes to permit and substantially prevent fluid flow through the valve 410 in first and second directions, respectively.

A second end 430 of the graft member 418 is attached to a circumferential support member 432 of the first support frame 412. The circumferential support member 432 defines a series of undulations 434. The second end 430 of the graft member 418 is attached to the circumferential support member 432 to substantially follow the series of undulations 434. As illustrated in FIG. 12, this configuration forms a series of leak paths 436 between the graft member 418 and an interior wall 482 of a body vessel 480 when the valve 410 is implanted in a body vessel 480. The leak paths 436 permit a controlled amount of fluid flow through the body vessel 480 at the location of the valve 410 without encountering the valve orifice 424.

The circumferential support member432 can have any suitable configuration, and the illustrated configuration is exemplary in nature. The circumferential support member 432 need only provide a configuration that facilitates formation of one or more leak paths between the graft member 418 and the interior vessel wall 482.

The graft member 418 can also include additional features that facilitate the passage of fluid flow that does not encounter the valve orifice 424, such as slits 438.

The graft member 418 is a flexible member and can be formed of any suitable material, including all materials described above for the valve members in other embodiments.

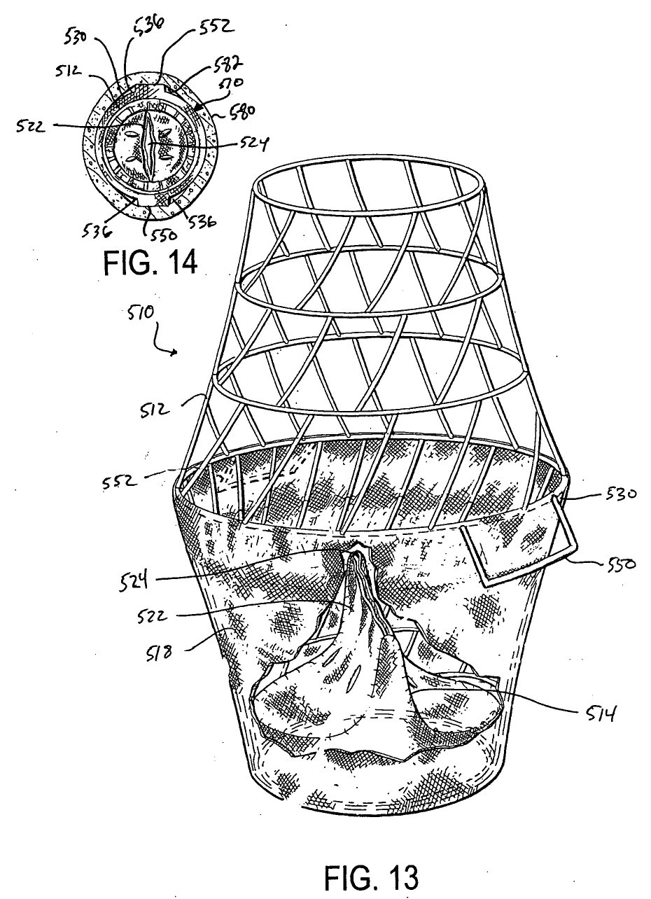

FIGS. 13 and 14 illustrate a medical device 510 according to a fifth exemplary embodiment. The medical device 510 according to this embodiment is similar to the embodiment illustrated in FIGS. 11 and 12, except as described below. Accordingly, the medical device 510 comprises a valve that includes first 512 and second 514 support frames, a tubular graft member 518 that forms a valve orifice 524 at one end 522 and is attached to the first support frame 512 at a second end 530.

In this embodiment, first 550 and second 552 spacing wings are disposed on the first support frame 512. As illustrated in FIG. 14, the spacing wings 550, 552 space the graft member 518 from an interior wall 582 of a body vessel 580 to form a series of leak paths 536 that permit a controlled amount of fluid flow through the body vessel 580 at the location of the valve 510 without encountering the valve orifice 524.

In the illustrated embodiment, the spacing wings 550, 552 are integrally formed by the wire member of the first support frame 512. The wings 550, 552 can, however, comprise separately attached members or have any other suitable configuration. Also, while the illustrated embodiment includes two spacing wings 550, 552, any suitable number of spacing wings can be used. The number chosen for a medical device according to a particular embodiment of the invention will depend on several considerations, including the quantity of fluid flow desired to pass through the body vessel without encountering the valve orifice 524.

In the tubular valve embodiments illustrated in FIGS. 11 through 14, it is understood that a single support frame and that the second support frame is an optional element. Also, the a substantially tubular graft member could be used instead of a tubular graft member. For example, two or more graft members could be arranged on the support frame to substantially create a tubularformation, despite their separate and distinct nature.

The leak path in any embodiment can enable flow from any suitable location or locations along a length of the medical device. The location(s) chosen for a medical device according to a particular embodiment will depend on several considerations, including the environment in which the medical device is intended to be placed. For example, venous valves that include one or more valve members that form pockets with the vessel wall may benefit from a leak path that enables retrograde flow from the pocket region of the device. Adaptations, such as the scallops illustrated in FIGS. 1 through 3 and the projections illustrated in FIGS. 4 through 8, can be positioned in the appropriate location to enable flow from the desired location. As another example, valve devices may also benefit from flow enabled from another location along the length of the device, such as a top or proximal region. FIGS. 9 and 10 illustrate an exemplary medical device in which retrograde flow is enabled from a location at the proximal end of the device. This structure can be used in conjunction with additional structure that enables flow from another location along the length of the device, such as a space between portions of the support frame at the distal end, as best illustrated in FIG. 9. Also, as best illustrated in FIGS. 11 through 14, a leak path can be used in conjunction with other flow-enabling features, such as openings and slits in valve members.

The inclusion of a leak path in medical devices in which a flexible material is used in the valve-forming element, such as the valve members illustrated in FIGS. 1 through 9 and the graft member illustrated in FIGS. 11 through 14, is particularly advantageous because the flexible material is likely to move intermittently and/or irregularly when the device is placed in a body vessel. This movement may create areas in which fluid is largely excluded from flushing action of normal flow, which could lead to stagnation and, in the case of blood vessels, thrombus formation. The leak paths can be positioned to provide a draining effect from such areas.

The foregoing detailed description provides exemplary embodiments of and includes the best mode for practicing the invention. These embodiments are intended only to serve as examples of the invention, and not to limit the scope of the invention, or its protection, in any manner.

What is claimed is:

1. An implantable medical device for regulating fluid flow through a body vessel, comprising:
a support frame having radially compressed and radially expanded configurations;
means forforming a leak path between the support frame and an interiorwall of said body vessel; and
at least one flexible valve member attached to the support frame, the valve member moveable between a first position that permits said fluid flow in a first direction and a second position that substantially prevents said fluid flow in a second direction.

2. The implantable medical device according to claim 1, wherein the means for forming a leak path between the support frame and an interior wall of said body vessel comprises a channel defined by a portion of the support frame.

3. The implantable medical device according to claim 2, wherein the channel has a cross-sectional shape that is substantially ovoid in shape.

4. The implantable medical device according to claim 2, wherein the channel includes an interior surface and further comprising a coating disposed on a portion of the interior surface.

5. The implantable medical device according to claim 4, wherein the coating comprises a thromboresistant or anti-thrombogenic coating.

6. The implantable medical device according to claim 5, wherein the coating comprises heparin.

7. The implantable medical device according to claim 1, wherein the means for forming a leak path between the support frame and an interior wall of said body vessel comprises a projection defined by a portion of the support frame and adapted to space a portion of the support frame from the interior wall of said body vessel.

8. The implantable medical device according to claim 1, wherein the means for forming a leak path between the support frame and an interior wall of said body vessel comprises a series of scalloped edges defined by a portion of the support frame.

9. The implantable medical device according to claim 1, wherein the means for forming a leak path between the support frame and an interior wall of said body vessel comprises a spacing wing disposed on a portion of the support frame, the spacing wing extending outward from the support frame and adapted to space a portion of the support frame from the interior wall of said body vessel.

10. The implantable medical device according to claim 1, wherein the valve member is formed of a bioremodelable material.

11. The implantable medical device according to claim 1, wherein the valve member is formed of an extracellular matrix material.

12. The implantable medical device according to claim 1, wherein the valve member is formed of small intestine submucosa.

13. The implantable medical device according to claim 1, wherein the valve member has first and second edges, the first edge attached to the support frame and the second edge being free of the support frame.

14. An implantable medical device for regulating fluid flow through a body vessel, comprising:
a support frame having radially compressed and radially expanded configurations;
means forforming a leak path between the support frame and an interiorwall of said body vessel; and
a first flexible valve member attached to the support frame and having first and second edges, the first edge attached to the support frame and the second edge being free of the support frame; and
a second flexible valve member attached to the support frame and having third and fourth edges, the third edge attached to the support frame and the fourth edge being free of the support frame;
wherein the second and fourth edges cooperatively defining a closeable valve opening through which said fluid flow can pass.

15. The implantable medical device according to claim 14, wherein the means for forming a leak path between the support frame and an interior wall of said body vessel comprises a channel defined by a portion of the support frame.

16. The implantable medical device according to claim 14, wherein the means for forming a leak path between the support frame and an interior wall of said body vessel comprises a projection defined by a portion of the support frame and adapted to space a portion of the support frame from the interior wall of said body vessel.

17. The implantable medical device according to claim 14, wherein the means for forming a leak path between the support frame and an interior wall of said body vessel comprises a series of scalloped edges defined by a portion of the support frame.

18. The implantable medical device according to claim 14, wherein the means for forming a leak path between the support frame and an interior wall of said body vessel comprises a spacing wing disposed on a portion of the support frame, the spacing wing extending outward from the support frame and adapted to space a portion of the support frame from the interior wall of said body vessel.

19. An implantable medical device for regulating fluid flow through a body vessel, comprising:
a support frame having radially compressed and radially expanded configurations and defining an interior space, the support frame defining adaptations that space a portion of the support frame from an interior wall of said body vessel to form a leak path between the support frame and an interior wall of said body vessel; and
a tubular graft member attached to the support frame and having first and second ends, the first end defining a valve orifice that permits said fluid flow in a first direction and a second position that substantially prevents said fluid flow in a second direction.

20. The implantable medical device according to claim 19, wherein the first end of the tubular graft member is inverted into the interior space of the support frame.
